(19) United States
(12) Patent Application Publication
(10) Pub. No.: US 2020/0163908 A1
(43) Pub. Date: May 28, 2020

(54) METHODS AND COMPOSITIONS FOR REGULATING GLUCOSE HOMEOSTASIS

(71) Applicant: ARIZONA BOARD OF REGENTS ON BEHALF OF THE UNIVERSITY OF ARIZONA, Tucson, AZ (US)

(72) Inventors: Benjamin J. Renquist, Tucson, AZ (US); Caroline E. Geisler, Tucson, AZ (US)

(21) Appl. No.: 16/617,108

(22) PCT Filed: May 25, 2018
(86) PCT No.: PCT/US18/34680

§ 371 Date: Nov. 26, 2019

Related U.S. Application Data
(60) Provisional application No. 62/647,468, filed on Mar. 23, 2018.
(60) Provisional application No. 62/511,753, filed on May 26, 2017.

(51) Int. Cl. A61K 31/145; A61K 31/197; A61P 3/04; A61P 3/10

(52) U.S. Cl. CPC A61K 31/145; A61P 3/10; A61P 3/04; A61K 31/197

(57) ABSTRACT

Methods and compositions (such as compounds, drugs, molecules, etc.) for regulating glucose homeostasis, for example for treating diabetes-related conditions such as hyperinsulinemia and insulin resistance. The methods and compositions herein may feature limiting hepatic mitochondrial uncoupling, decreasing hepatic GABA release, decreasing hepatic GABA synthesis, and/or maintaining hepatocyte membrane potential. More specifically, the methods and compositions herein may feature inhibitors for GABA synthesis and/or inhibitors for GABA release, e.g., inhibitors for GABA-T, BGT1 (GABA transporter), GAT2 (GABA transporter), M3R, etc. The present invention also features altering food intake by regulating GABA production or GABA release.

### CROSS REFERENCE

This application claims priority to U.S. Provisional Patent Application No. 62/511,753 filed May 26, 2017, and U.S. Provisional Patent Application No. 62/647,468 filed Mar. 23, 2018, the specification(s) of which is/are incorporated herein in their entirety by reference.

### FIELD OF THE INVENTION

The present invention relates to type II diabetes, insulin resistance, hyperinsulinemia, and obesity-related conditions. For example, the present invention relates to (though is not limited to) methods and compositions for treating hyperinsulinemia and insulin resistance.

### BACKGROUND OF THE INVENTION

Type II diabetes (T2D) is a global health concern that affects 30 million Americans, doubling both the risk of death and medical costs for an individual. Non-alcoholic fatty liver disease (NAFLD) is strongly associated with an increased risk of developing diabetes, while the degree of hepatic steatosis is directly related to the severity of systemic insulin resistance, glucose intolerance, and hyperinsulinemia.

The hepatic vagal nerve acts as a conduit by which the liver communicates nutritional status to affect pancreatic insulin release and peripheral tissue insulin sensitivity. The hepatic vagal afferent nerve (HVAN) regulates parasympathetic efferent nerve activity at the pancreas to alter insulin secretion. A decrease in HVAN firing frequency stimulates insulin release, conversely increased HVAN firing frequency decreases serum insulin. The HVAN also regulates whole-body insulin sensitivity. Hepatic vagotomy diminishes insulin sensitivity and skeletal muscle glucose clearance in insulin sensitive rats, while improving insulin sensitivity and glucose tolerance in insulin resistant mice. Therefore, the firing frequency of the HVAN is integral to controlling insulin secretion and sensitivity.

Inventors have surprisingly discovered a mechanism by which hepatic steatosis induces systemic insulin dysregulation, while establishing that hepatocytes release GABA in a manner regulated by hepatocyte membrane potential. This model explains how obesity and fasting can both induce hepatic lipid accumulation, yet only obesity causes hyperinsulinemia. Moreover, the model provides a framework to explain how portal glucose delivery, known to decrease HVAN activity, decreases skeletal muscle glucose clearance and encourages hepatic glucose clearance.

### SUMMARY OF THE INVENTION

The present invention features methods and compositions for regulating glucose homeostasis. Briefly, the methods and compositions herein may feature limiting hepatic mitochondrial uncoupling, decreasing hepatic GABA release, and preventing obesity induced depolarization of the hepatocyte membrane potential. More specifically, the methods may feature inhibitors for GABA synthesis and/or inhibitors for GABA release, e.g., inhibitors for GABA-T, BGT1 (GABA transporter), GAT2 (GABA transporter), M3R, etc. The methods and compositions herein may be used for a variety of purposes including but not limited to treating type II diabetes, insulin resistance, hyperinsulinemia, hypertension, etc.

The present invention also features altering food intake by regulating GABA production or GABA release. For example, the present invention features methods and compositions for losing weight (reducing food intake) by depressing hepatic GABA production or release. The present invention also features methods and compositions for gaining weight (increasing food intake) by enhancing hepatic GABA production or release.

The methods and compositions (e.g., compounds, drugs, molecules, e.g., siRNA, etc.) herein may be used for treating obesity related complications such as but not limited to diabetes (e.g., type II diabetes) and hypertension. For example, the present invention features methods for treating obesity-related complications using compositions (e.g., compounds, drugs, molecules, e.g., siRNA, etc.) that inhibit the activity or expression of (or silence) GABA-transaminase, hepatic succinate semialdehyde dehydrogenase, hepatic aspartate aminotransferase, malate dehydrogenase, aspartate aminotransferase, BGT1 (protein encoded for by SLC6A12), GAT2 (protein encoded for by SLC6A13), UCP2, the like, or a combination thereof. The present invention also features methods for treating obesity-related complications by hyperpolarizing liver cells or by preventing obesity induced depolarization of liver cells. In some embodiments, the compositions of the present invention improve insulin sensitivity and glucose clearance, decrease blood glucose and insulin concentrations, and/or decrease/normalize blood pressure.

Again, more specifically, the present invention features methods and compositions for treating obesity-related conditions by limiting hepatic GABA-Transaminase, succinate semialdehyde dehydrogenase, malate dehydrogenase, aspartate aminotransferase, BGT1 (protein encoded for by SLC6A12), GAT2 (protein encoded for by SLC6A13) or UCP2 activity or expression; inhibiting hepatic GABA release; increasing hepatic Aspartate release; hyperpolarizing the hepatocyte/preventing the obesity induced depolarization of the hepatocyte; preventing GABA signaling on the hepatic vagal afferent nerve; increasing Aspartate signaling on the hepatic vagal afferent nerve; blocking muscarinic 3 receptor signaling on the beta and alpha cell; blocking pancreatic parasympathetic efferent signaling; increasing muscarinic receptor signaling on endothelial cells in the vasculature to limit vasoconstriction/encourage vasodilation; enhancing skeletal muscle parasympathetic efferent signaling; and the like.

As previously discussed, the present invention features methods of treating an obesity-related condition in a subject in need thereof. In certain embodiments, the method comprises administering to the subject a therapeutic amount of a composition for decreasing hepatic GABA synthesis or hepatic GABA release, wherein decreasing hepatic GABA synthesis or hepatic GABA release decreases blood glucose and improves insulin sensitivity. In certain embodiments, the composition prevents obesity-induced depolarization of hepatocytes. In certain embodiments, the composition normalizes blood pressure. In certain embodiments, the composition reduces hepatic mitochondrial uncoupling.

In certain embodiments, the composition comprises an inhibitor of GABA-T. In certain embodiments, the composition comprises an inhibitor of BGT1. In certain embodiments, the composition comprises an inhibitor of GAT2. In certain embodiments, the composition comprises an inhibitor of M3R for inhibiting insulin release. In certain embodiments, the composition comprises an activator of M3R for improving insulin sensitivity and stimulating insulin release. In certain embodiments, the composition comprises an inhibitor of UCP2. In certain embodiments, the composition comprises an inhibitor of hepatic succinate semialdehyde dehydrogenase. In some embodiments, the composition comprises an inhibitor of GHB production. In some embodiments, the composition comprises an inhibitor of GHB conversion to succinate semialdehyde (SSA). In some embodiments, the composition comprises a GHB dehydrogenase inhibitor.

In certain embodiments, the composition is a drug, a compound, or a molecule. In certain embodiments, the molecule is an anti-sense oligonucleotide. In certain embodiments, the obesity-related condition is diabetes, hyperglycemia, insulin resistance, glucose intolerance, or hypertension. In certain embodiments, the composition inhibits GABA signaling on the hepatic vagal afferent nerve.

In certain embodiments, the composition causes a fasting blood glucose of 120 mg/dL or less. In certain embodiments, the composition causes a fasting blood glucose of 110 mg/dL or less. In certain embodiments, the composition causes a fasting blood glucose of 100 mg/dL or less. In certain embodiments, the composition causes a fasting blood glucose of 90 mg/dL or less. In certain embodiments, the composition causes a fasting blood glucose from 90 mg/dL to 100 mg/dL. In certain embodiments, the composition causes a fasting insulin level of 5 mmol/mL or less. In certain embodiments, the composition causes a fasting insulin level of 10 mmol/mL or less. In certain embodiments, the composition causes a fasting insulin level from 2 to 10 mmol/mL.

In certain embodiments, the composition comprises ethanolamine-O-sulfate (EOS). In certain embodiments, the composition comprises vigabatrin. In certain embodiments, the composition does not cross the blood-brain barrier. In certain embodiments, the composition comprises a derivative of vigabatrin or EOS that does not cross the blood-brain barrier.

The present invention also features methods for improving insulin sensitivity in a subject in need thereof. In certain embodiments, method comprises administering to the subject a therapeutic amount of a composition for decreasing hepatic GABA synthesis or hepatic GABA release, wherein decreasing hepatic GABA synthesis or hepatic GABA release improves insulin sensitivity. In certain embodiments, the composition restores insulin sensitivity to that of a non-diabetic individual.

The present invention also features methods for improving insulin sensitivity and limiting hyperinsulinemia in a subject in need thereof. In some embodiments, the method comprises administering to the subject a therapeutic amount of a composition for decreasing hepatic GABA synthesis or hepatic GABA release, wherein decreasing hepatic GABA synthesis or release improves insulin sensitivity and decreases hyperinsulinemia.

In certain embodiments, the composition comprises an inhibitor of GABA-T. In certain embodiments, the composition comprises an inhibitor of BGT1. In certain embodiments, the composition comprises an inhibitor of GAT2. In certain embodiments, the composition comprises an inhibitor of M3R for inhibiting insulin release. In certain embodiments, the composition comprises an activator of M3R for improving insulin sensitivity and stimulating insulin release. In certain embodiments, the composition comprises an inhibitor of UCP2. In certain embodiments, the composition comprises an inhibitor of hepatic succinate semialdehyde dehydrogenase. In some embodiments, the composition comprises an inhibitor of GHB production. In some embodiments, the composition comprises an inhibitor of GHB conversion to succinate semialdehyde (SSA). In some embodiments, the composition comprises a GHB dehydrogenase inhibitor.

In certain embodiments, the composition is a drug, a compound, or a molecule. In certain embodiments, the molecule is an anti-sense oligonucleotide. In certain embodiments, the composition inhibits GABA signaling on the hepatic vagal afferent nerve.

In certain embodiments, the composition causes a fasting blood glucose of 110 mg/dL or less. In certain embodiments, the composition causes a fasting blood glucose of 100 mg/dL or less. In certain embodiments, the composition causes a fasting blood glucose of 90 mg/dL or less. In certain embodiments, the composition causes a fasting blood glucose from 90 mg/dL to 100 mg/dL. In certain embodiments, the composition causes a fasting insulin level of 5 mmol/mL or less. In certain embodiments, the composition causes a fasting insulin level of 10 mmol/mL or less. In certain embodiments, the composition causes a fasting insulin level from 2 to 10 mmol/mL.

In certain embodiments, the composition comprises ethanolamine-O-sulfate (EOS). In certain embodiments, the composition comprises vigabatrin. In certain embodiments, the composition does not cross the blood-brain barrier. In certain embodiments, the composition comprises a derivative of vigabatrin or EOS that does not cross the blood-brain barrier.

The present invention also features a pharmaceutical composition for treating an obesity-related condition, wherein the composition is effective to decrease blood glucose, decrease blood insulin, improve insulin sensitivity, increase glucose tolerance, and decrease/normalize blood pressure or a combination thereof.

In certain embodiments, the composition comprises an inhibitor of a GABA transporter. In certain embodiments, the inhibitor of the GABA transporter inhibits BGT1, GAT2, or both.

In certain embodiments, the composition comprises an inhibitor of M3R for inhibiting insulin release. In certain embodiments, the composition comprises an activator of M3R for improving insulin sensitivity and stimulating insulin release. In certain embodiments, the composition comprises an inhibitor of UCP2. In certain embodiments, the composition comprises an inhibitor of hepatic succinate semialdehyde dehydrogenase. In some embodiments, the composition comprises an inhibitor of GHB production. In some embodiments, the composition comprises an inhibitor of GHB conversion to succinate semialdehyde (SSA). In some embodiments, the composition comprises a GHB dehydrogenase inhibitor.

In certain embodiments, the composition is a drug, a compound, or a molecule. In certain embodiments, the molecule is an anti-sense oligonucleotide. In certain embodiments, the composition inhibits GABA signaling on the hepatic vagal afferent nerve.

The present invention also features methods for causing a subject in need thereof to lose weight. In certain embodiments, the method comprises: administering to the patient a therapeutic amount of a composition for decreasing hepatic GABA synthesis or hepatic GABA release, wherein decreasing hepatic GABA synthesis or hepatic GABA release causes a decrease in food intake so that the subject loses weight. In certain embodiments, the composition prevents obesity-induced depolarization of hepatocytes. In certain embodiments, the composition normalizes blood pressure. In certain embodiments, the composition reduces hepatic mitochondrial uncoupling.

In certain embodiments, the composition comprises an inhibitor of GABA-T. In certain embodiments, the composition comprises an inhibitor of BGT1. In certain embodiments, the composition comprises an inhibitor of GAT2. In certain embodiments, the composition comprises an inhibitor of M3R for inhibiting insulin release. In certain embodiments, the composition comprises an activator of M3R for improving insulin sensitivity and stimulating insulin release. In certain embodiments, the composition comprises an inhibitor of UCP2. In certain embodiments, the composition comprises an inhibitor of hepatic succinate semialdehyde dehydrogenase. In some embodiments, the composition comprises an inhibitor of GHB production. In some embodiments, the composition comprises an inhibitor of GHB conversion to succinate semialdehyde (SSA). In some embodiments, the composition comprises a GHB dehydrogenase inhibitor.

In certain embodiments, the composition is a drug, a compound, or a molecule. In certain embodiments, the molecule is an anti-sense oligonucleotide. In certain embodiments, the composition inhibits GABA signaling on the hepatic vagal afferent nerve.

In certain embodiments, the composition causes a fasting blood glucose of 110 mg/dL or less. In certain embodiments, the composition causes a fasting blood glucose of 100 mg/dL or less. In certain embodiments, the composition causes a fasting blood glucose of 90 mg/dL or less. In certain embodiments, the composition causes a fasting blood glucose from 90 mg/dL to 100 mg/dL. In certain embodiments, the composition causes a fasting insulin level of 5 mmol/mL or less. In certain embodiments, the composition causes a fasting insulin level of 10 mmol/mL or less. In certain embodiments, the composition causes a fasting insulin level from 2 to 10 mmol/mL.

In certain embodiments, the composition comprises ethanolamine-O-sulfate (EOS). In certain embodiments, the composition comprises vigabatrin. In certain embodiments, the composition does not cross the blood-brain barrier. In certain embodiments, the composition comprises a derivative of vigabatrin or EOS that does not cross the blood-brain barrier.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE DRAWINGS

The features and advantages of the present invention will become apparent from a consideration of the following detailed description presented in connection with the accompanying figures in which:

FIG. 1A shows ligand-induced change in hepatocyte membrane potential in mice treated with a virus encoding a cre-dependent depolarizing channel. Data in FIG. 1A was collected concurrently with data in FIG. 1B. WT=wild type. * Denotes significant differences (P<0.05) between groups within that time point. All data are presented as mean±SEM.

FIG. 1B shows depolarizing ligand induced relative change in hepatic vagal afferent nerve activity. Data in FIG. 1A was collected concurrently with data in FIG. 1B. WT=wild type. * Denotes significant differences (P<0.05) between groups within that time point. All data are presented as mean±SEM.

FIG. 2A shows changes in serum insulin from ligand-dependent hepatocyte depolarization. Mice were fed and fasted albumin-cre mice 15 minutes after saline or depolarizing ligand (30 mg/kg) administration. All mice had previously been given a tail-vein injection of an adeno-associated virus encoding for a cre-dependent ligand-gated depolarizing channel and studies were performed after a minimum of 5 days post-injection. NS—non-significant. Number inside bar denotes n per group. All data are presented as mean±SEM.

FIG. 2B shows changes in serum glucose from ligand-dependent hepatocyte depolarization. Mice were fed and fasted albumin-cre mice 15 minutes after saline or depolarizing ligand (30 mg/kg) administration. All mice had previously been given a tail-vein injection of an adeno-associated virus encoding for a cre-dependent ligand-gated depolarizing channel and studies were performed after a minimum of 5 days post-injection. NS—non-significant. Number inside bar denotes n per group. All data are presented as mean±SEM.

FIG. 2C shows changes in glucose:insulin ratio from ligand-dependent hepatocyte depolarization. Mice were fed and fasted albumin-cre mice 15 minutes after saline or depolarizing ligand (30 mg/kg) administration. All mice had previously been given a tail-vein injection of an adeno-associated virus encoding for a cre-dependent ligand-gated depolarizing channel and studies were performed after a minimum of 5 days post-injection. NS—non-significant. Number inside bar denotes n per group. All data are presented as mean±SEM.

FIG. 2D shows changes in serum insulin from ligand-dependent hepatocyte depolarization. Mice were fed and fasted wild type mice 15 minutes after saline or depolarizing ligand (30 mg/kg) administration. All mice had previously been given a tail-vein injection of an adeno-associated virus encoding for a cre-dependent ligand-gated depolarizing channel and studies were performed after a minimum of 5 days post-injection. NS—non-significant. Number inside bar denotes n per group. All data are presented as mean±SEM.

FIG. 2E shows changes in serum glucose from ligand-dependent hepatocyte depolarization. Mice were fed and fasted wild type mice 15 minutes after saline or depolarizing ligand (30 mg/kg) administration. All mice had previously been given a tail-vein injection of an adeno-associated virus encoding for a cre-dependent ligand-gated depolarizing channel and studies were performed after a minimum of 5 days post-injection. NS—non-significant. Number inside bar denotes n per group. All data are presented as mean±SEM.

FIG. 2F shows changes in glucose:insulin ratio from ligand-dependent hepatocyte depolarization. Mice were fed and fasted wild type mice 15 minutes after saline or depolarizing ligand (30 mg/kg) administration. All mice had previously been given a tail-vein injection of an adeno-associated virus encoding for a cre-dependent ligand-gated depolarizing channel and studies were performed after a minimum of 5 days post-injection. NS—non-significant. Number inside bar denotes n per group. All data are presented as mean±SEM.

FIG. 2G shows serum insulin in fed wild type mice expressing a thyroxine binding globulin promoter driven depolarizing channel injected with either saline or ligand 10 minutes prior to an oral glucose load (2.5 g/kg). NS—non-significant. Number inside bar denotes n per group. All data are presented as mean±SEM.

FIG. 2H shows glucose in fed wild type mice expressing a thyroxine binding globulin promoter driven depolarizing channel injected with either saline or ligand 10 minutes prior to an oral glucose load (2.5 g/kg). NS—non-significant. Number inside bar denotes n per group. All data are presented as mean±SEM.

FIG. 2I shows glucose:insulin ratio in fed wild type mice expressing a thyroxine binding globulin promoter driven depolarizing channel injected with either saline or ligand 10 minutes prior to an oral glucose load (2.5 g/kg). NS—non-significant. Number inside bar denotes n per group. All data are presented as mean±SEM.

FIG. 3A shows hepatic UPC2 knockdown does not affect HFD-induced weight gain. All tests were performed after 8-10 weeks of HFD feeding. All data are presented as mean±SEM.

FIG. 3B shows the effect of hepatic UPC2 knockout on serum insulin. All tests were performed after 8-10 weeks of HFD feeding. All data are presented as mean±SEM. ^{a,b} Bars that do not share a common letter differ significantly (P<0.05; number inside bar denotes n).

FIG. 3C shows the effect of hepatic UPC2 knockout on glucose. All tests were performed after 8-10 weeks of HFD feeding. All data are presented as mean±SEM. NS—non-significant. ^{a,b} Bars that do not share a common letter differ significantly (P<0.05; number inside bar denotes n).

FIG. 3D shows the effect of hepatic UPC2 knockout on glucose:insulin ratio. All tests were performed after 8-10 weeks of HFD feeding. All data are presented as mean±SEM. ^{a,b} Bars that do not share a common letter differ significantly (P<0.05; number inside bar denotes n).

FIG. 3E shows the effect of hepatic UPC2 knockout on oral glucose tolerance (OGTT). All tests were performed after 8-10 weeks of HFD feeding. All data are presented as mean±SEM.

FIG. 3F shows the effect of hepatic UPC2 knockout on oral glucose tolerance (OGTT) area under the curve (AUC). All tests were performed after 8-10 weeks of HFD feeding. All data are presented as mean±SEM. NS—non-significant. ^{a,b} Bars that do not share a common letter differ significantly (P<0.05; number inside bar denotes n).

FIG. 3G shows the effect of hepatic UPC2 knockout on oral glucose stimulated serum insulin. All tests were performed after 8-10 weeks of HFD feeding. All data are presented as mean±SEM. NS—non-significant. ^{a,b} Bars that do not share a common letter differ significantly (P<0.05; number inside bar denotes n).

FIG. 3H shows the effect of hepatic UPC2 knockout on Insulin tolerance (ITT). All tests were performed after 8-10 weeks of HFD feeding. All data are presented as mean±SEM.

FIG. 3I shows the effect of hepatic UPC2 knockout on insulin tolerance (ITT) area under the curve (AUC). All tests were performed after 8-10 weeks of HFD feeding. All data are presented as mean±SEM. ^{a,b} Bars that do not share a common letter differ significantly (P<0.05; number inside bar denotes n).

FIG. 3J shows the effect of hepatic UPC2 knockout on the serum insulin response to the α2 adrenergic antagonist, Atimepazole. All tests were performed after 8-10 weeks of HFD feeding. All data are presented as mean±SEM. ^{a,b} Bars that do not share a common letter differ significantly (P<0.05; number inside bar denotes n).

FIG. 3K shows the effect of hepatic UPC2 knockout on muscarinic agonist Carbachol stimulated changes in serum insulin. All tests were performed after 8-10 weeks of HFD feeding. NS—non-significant. All data are presented as mean±SEM. ^{a,b} Bars that do not share a common letter differ significantly (P<0.05; number inside bar denotes n).

FIG. 4A shows liver specific expression of the Kir2.1 hyperpolarizing channel in a wild type mouse. Fluorescent imaging for red=tdTomato and blue=DAPI (nucleus).

FIG. 4B shows barium-induced change in hepatocyte membrane potential in Kir2.1 and eGFP (control) expressing mice. Number inside bar denotes n per group. * denotes significance (P<0.05) between Kir2.1 and controls. All data are presented as mean±SEM.

FIG. 4C shows hepatic Kir2.1 expression effects on HFD induced weight gain. * denotes significance (P<0.05) between Kir2.1 and controls. All data are presented as mean±SEM.

FIG. 4D shows hepatic Kir2.1 expression effects on serum insulin at 0, 3, 6, and 9 weeks. ^{a,b} Bars that do not share a common letter differ significantly (P<0.05; number inside bar denotes n per group). * denotes significance (P<0.05) between Kir2.1 and controls. All data are presented as mean±SEM.

FIG. 4E shows hepatic Kir2.1 expression effects on glucose at 0, 3, 6, and 9 weeks. ^{a,b} Bars that do not share a common letter differ significantly (P<0.05; number inside bar denotes n per group). * denotes significance (P<0.05) between Kir2.1 and controls. All data are presented as mean±SEM.

FIG. 4F shows hepatic Kir2.1 expression effects on glucose:insulin ratio at 0, 3, 6, and 9 weeks. NS—non-significant. ^{a,b} Bars that do not share a common letter differ significantly (P<0.05; number inside bar denotes n per group). * denotes significance (P<0.05) between Kir2.1 and controls. All data are presented as mean±SEM.

FIG. 4G shows the effect of hepatic Kir2.1 expression on oral glucose tolerance (OGTT) after 9 weeks of HFD feeding. * denotes significance (P<0.05) between Kir2.1 and controls. All data are presented as mean±SEM.

FIG. 4H shows the effect of hepatic Kir2.1 expression on OGTT area under the curve (AUC) after 9 weeks of HFD feeding. ^{a,b} Bars that do not share a common letter differ significantly (P<0.05; number inside bar denotes n per group). * denotes significance (P<0.05) between Kir2.1 and controls. All data are presented as mean±SEM.

FIG. 4I shows the effect of hepatic Kir2.1 expression on oral glucose stimulated serum insulin after 9 weeks of HFD feeding. NS—non-significant. ^{a,b} Bars that do not share a common letter differ significantly (P<0.05; number inside bar denotes n per group). * denotes significance (P<0.05) between Kir2.1 and controls. All data are presented as mean±SEM.

FIG. 4J shows the effect of hepatic Kir2.1 expression on insulin tolerance (ITT) after 9 weeks of HFD feeding. * denotes significance (P<0.05) between Kir2.1 and controls. All data are presented as mean±SEM.

FIG. 4K shows the effect of hepatic Kir2.1 expression on ITT AUC after 9 weeks of HFD feeding. ^{a,b} Bars that do not share a common letter differ significantly (P<0.05; number inside bar denotes n per group). * denotes significance (P<0.05) between Kir2.1 and controls. All data are presented as mean±SEM.

FIG. 4L shows the effect of an α2 adrenergic antagonist, Atimepazole, on serum insulin in control and hepatic Kir2.1 expressing mice after 9 weeks of HFD feeding on. ^{a,b} Bars that do not share a common letter differ significantly (P<0.05; number inside bar denotes n per group). * denotes significance (P<0.05) between Kir2.1 and controls. All data are presented as mean±SEM.

FIG. 4M shows the effect of the muscarinic agonist, Carbachol, on serum insulin in control and hepatic Kir2.1 expressing mice after 9 weeks of HFD feeding. ^{a,b} Bars that do not share a common letter differ significantly (P<0.05; number inside bar denotes n per group). * denotes significance (P<0.05) between Kir2.1 and controls. All data are presented as mean±SEM.

FIG. 4N shows the effect of the muscarinic antagonist, methylatropine bromide, on serum insulin in control and hepatic Kir2.1 expressing mice after 9 weeks of HFD. NS—non-significant. ^{a,b} Bars that do not share a common letter differ significantly (P<0.05; number inside bar denotes n per group). * denotes significance (P<0.05) between Kir2.1 and controls. All data are presented as mean±SEM.

FIG. 5A shows body weight during treatment: mice were fed a high fat-high sucrose diet for 8-10 weeks to induce obesity then treated with GABA-Transaminase inhibitors ethanolamine-O-sulfate (EOS) or vigabatrin (8 mg/day), or PBS (control). NS—non-significant. ^{a,b} Bars that do not share a common letter differ significantly within day (P<0.05; number inside bar denotes n per group). *denotes significance (P<0.05) within treatment group comparing before and during treatment. All data are presented as mean±SEM.

FIG. 5B shows basal serum insulin on treatment day 4 of the experiment in FIG. 5A.

FIG. 5C shows glucose on treatment day 4 of the experiment of FIG. 5A.

FIG. 5D shows glucose:insulin ratio on treatment day 4 of the experiment in FIG. 5A.

FIG. 5E shows oral glucose tolerance (OGTT) on treatment day 4 of the experiment of FIG. 5A.

FIG. 5F shows OGTT area under the curve (AUC) on treatment day 4 of the experiment of FIG. 5A.

FIG. 5G shows oral glucose glucose stimulated serum insulin on treatment day 4 of the experiment of FIG. 5A.

FIG. 5H shows insulin tolerance (ITT) on treatment day 4 of the experiment of FIG. 5A.

FIG. 5I shows ITT AUC on treatment day 4 of the experiment of FIG. 5A.

FIG. 5J shows a muscarinic antagonist (methylatropine-bromide) injection on treatment day 5 of the experiment of FIG. 5A.

FIG. 5K shows GABA release (μmol/mg DNA) from hepatic slices is increased by obesity and inhibited by Kir2.1 expression. Hepatic slices were collected from lean, obese, and obese Kir2.1 expressing mice.

FIG. 5L shows aspartate release (μmol/mg DNA) is decreased in obesity and not affected by Kir2.1 expression. Hepatic slices were collected from lean, obese, and obese Kir2.1 expressing mice.

FIG. 5M shows obesity increases GABA-Transaminase mRNA expression, which is not affected by Kir2.1 expression. Hepatic slices were collected from lean, obese, and obese Kir2.1 expressing mice.

FIG. 5N shows bath application of the GABA-T inhibitor, ethanolamine-O-sulfate (EOS), decreased GABA release from slices from obese mice. Hepatic slices were collected from lean, obese, and obese Kir2.1 expressing mice.

FIG. 6A shows that hepatic vagotomized mice gain less weight on a high fat diet than sham surgery mice. All data are presented as mean±SEM

FIG. 6B shows that hepatic vagotomy limits hyperinsulinemia at 9 weeks of high fat feeding. All data are presented as mean±SEM

FIG. 6C shows that neither high fat feeding diet nor hepatic vagotomy affected serum glucose. All data are presented as mean±SEM

FIG. 6D shows the serum glucose:insulin ratio, indicative of insulin sensitivity, was elevated by hepatic vagotomy both in chow fed mice and mice on a high fat diet for 9 weeks. All data are presented as mean±SEM

FIG. 6E shows that hepatic vagotomy did not affect oral glucose tolerance test.

FIG. 6F shows that hepatic vagotomy did not affect OGTT AUC. All data are presented as mean±SEM

FIG. 6G shows that hepatic vagotomy limits oral glucose stimulated insulin release. All data are presented as mean±SEM

FIG. 6H shows that hepatic vagotomy improves insulin tolerance. All data are presented as mean±SEM

FIG. 6I shows that hepatic vagotomy improves insulin tolerance as observed by the ITT AUC. All data are presented as mean±SEM

FIG. 7A shows that GABA export is Na+ dependent. By decreasing extracellular Na+, GABA export from liver slices is encouraged. Experiments were done in lean mice.

FIG. 7B shows that GAT2 (inhibited by nipoetic acid) and BGT1 (inhibited by betaine) transport GABA out of the liver slide. Experiments were done in lean mice.

FIG. 8A shows systolic blood pressure in mice with an intact hepatic vagal nerve.

FIG. 8B shows systolic blood pressure in mice with a hepatic vagotomy.

FIG. 8C shows diastolic blood pressure in mice with an intact hepatic vagal nerve.

FIG. 8D shows systolic blood pressure in mice with a hepatic vagotomy.

FIG. 8E shows mean blood pressure in mice with an intact hepatic vagal nerve.

FIG. 8F shows mean blood pressure in mice with a hepatic vagotomy.

FIG. 8G shows heart rate in mice with an intact hepatic vagal nerve.

FIG. 8H shows heart rate in mice with a hepatic vagotomy.

FIG. 9 shows a schematic view of possible hepatic control of insulin secretion and sensitivity. Obesity induced hepatic lipid accumulation depolarizes the hepatocyte resulting in a decrease in hepatic afferent vagal nerve (HVAN) activity. (1) β-oxidation depresses the mitochondrial NAD⁺:NADH₂ and FAD⁺:FADH₂ ratios driving succinate to succinate semialdehyde, generating substrate for GABA-Transaminase. (2) GABA-Transaminase produces GABA and α-ketoglutarate, a substrate for aspartate aminotransferase. (3) Increased gluconeogenic flux drives the mitochondrial export of OAA as malate, and (4) released GABA acts on GABA_{A} receptors to hyperpolarize the HVAN. (5) This decreased HVAN activity increases pancreatic vagal efferent acetylcholine release and muscarinic 3 receptor (M3R) signaling at 3-cells. When the beta-cell is depolarized by glucose, as occurs in obesity, this increased acetylcholine signaling stimulates insulin release. (6) It is proposed that the hepatic lipid accumulation and hepatocyte depolarization induced depression of HVAN activity decreases insulin sensitivity at skeletal muscle. Abbreviations: OAA=oxaloacetate, AST=aspartate aminotransferase, GABA-T=GABA-Transaminase, α-KG=α-ketoglutarate, SSADH=succinate semialdehyde dehydrogenase.

### DETAILED DESCRIPTION OF THE INVENTION

### Hepatocyte Depolarization Depresses HVAN Firing Activity

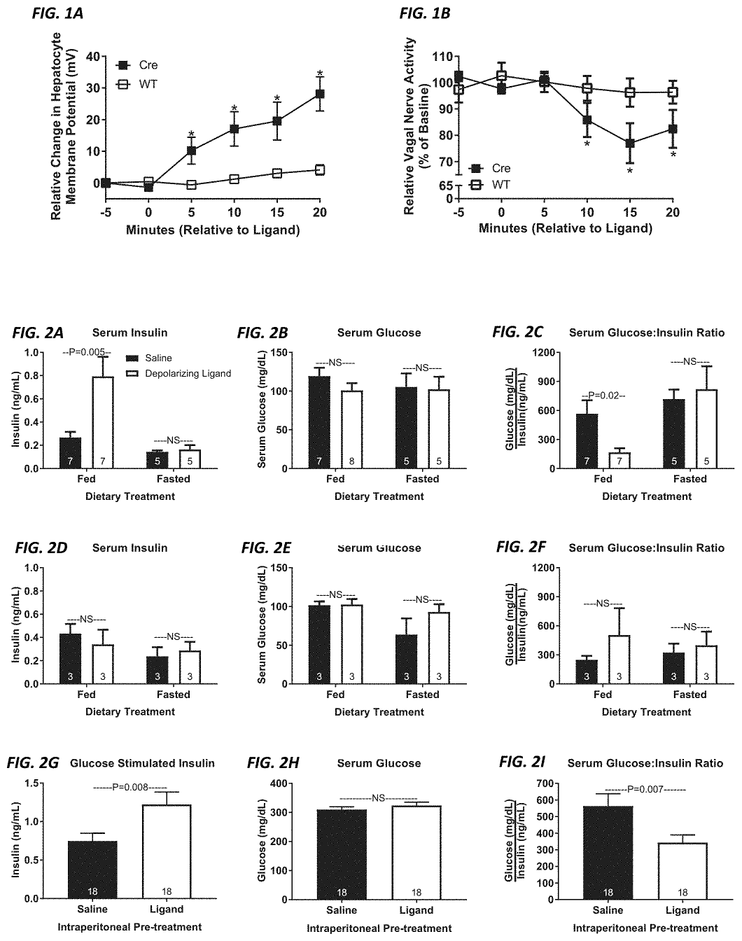

To investigate if hepatocyte depolarization affects HVAN firing activity, a genetically engineered, ligand-gated depolarizing ion channel was used. An adeno-associated virus serotype 8 (AAV8) encoding this ligand-gated depolarizing channel and green fluorescent protein (eGFP) flanked by LoxP sites was intravenously delivered to wild type mice or mice expressing cre-recombinase driven by the albumin promoter. Liver-specific channel expression in albumin-cre expressing mice and no expression in wild type mice was confirmed. Hepatocyte membrane potential and HVAN activity were simultaneously measured in the anesthetized mouse to assess the influence of hepatocyte depolarization on HVAN firing activity. Bath application of the ligand depolarized hepatocytes and decreased HVAN firing activity in albumin-cre, channel expressing mice (see FIG. 1A, FIG. 1B). There was no effect on either hepatocyte membrane potential or HVAN in wild type mice (see FIG. 1A, FIG. 1B). FIG. 1A and FIG. 1B together show that acute hepatocyte depolarization depresses hepatic vagal afferent nerve activity.

### Acute Hepatic Depolarization Elevates Serum Insulin

Parasympathetic nervous system release of acetylcholine onto β-cell muscarinic 3 receptors (M3R) is essential for glucose stimulated insulin secretion. Hepatocyte depolarization depresses HVAN activity (FIG. 1A, FIG. 1B), which increases acetylcholine release from parasympathetic efferent nerves onto the pancreas, enhancing insulin secretion from β-cells. Administration of the ligand more than doubled serum insulin in albumin-cre mice, which express the ligand activated depolarizing channel, without affecting serum glucose concentrations (FIG. 2A, FIG. 2B), indicating hepatocyte depolarization causes hyperinsulinemia. Accordingly, ligand decreased the glucose:insulin ratio in albumin-cre mice (FIG. 2C).

The β-cell insulin secretory response to acetylcholine depends on circulating glucose concentrations. Acetylcholine signaling through M3R stimulates insulin release when the β-cell is simultaneously depolarized by glucose. Yet, under fasted, hypoglycemic conditions, acetylcholine release at the R-cell increases the readily releasable pool of insulin in preparation for the next meal. In fasted albumin-cre, channel expressing mice, ligand did not affect serum insulin, glucose, or the glucose: insulin ratio (FIG. 2A, FIG. 2B, FIG. 2C). Notably, ligand did not alter serum insulin, glucose, or the glucose:insulin ratio in either fed or fasted wild type mice (Figs. FIG. 2D, FIG. 2E, FIG. 2F).

A second model of hepatocyte depolarization in which liver specific expression of the same ligand-gated depolarizing channel was independent of cre-recombinase and instead driven by the thyroxine binding globulin (TBG) promoter was developed. To ensure stimulatory concentrations of circulating glucose, an oral glucose gavage (2.5 g/kg body weight) was given 10 minutes following IP ligand injection. As previously observed, ligand administration elevated serum insulin and lowered the glucose:insulin ratio in mice expressing the depolarizing channel (FIG. 2G, FIG. 2H, FIG. 2I). Ligand injection did not affect the rise in serum glucose following an oral gavage of glucose (FIG. 2H).

### Hepatic UCP2 Knockout Protects Against Diet-Induced Hyperinsulinemia and Insulin Resistance

Hepatic lipids activate the transcription factor, peroxisome proliferator activated receptor (PPARα), to promote flux through gluconeogenesis and ketogenesis. Incredibly, PPARα knockout mice are protected from diet induced insulin resistance and hyperinsulinemia. Hepatic vagotomy enhances peripheral insulin action in obese wild type mice, but not in mice that lack PPARα expression. Hepatic uncoupling protein 2 (UCP2), a PPARα target gene, is upregulated in diabetes and obesity. While this adaptation initially protects against lipotoxicity, chronic elevation of UCP2 disrupts cellular metabolism and depletes hepatic ATP by uncoupling mitochondrial electron transport chain activity from ATP synthesis. Type II diabetics have lower hepatic ATP concentrations, and both peripheral and hepatic insulin sensitivity is significantly correlated with liver ATP concentrations.

Hepatic specific UCP2 knockout mice were generated. Elimination of hepatic UCP2 (UCP2 KO) had no effect on serum insulin, glucose, the glucose:insulin ratio, glucose clearance, glucose stimulated serum insulin, or insulin sensitivity in chow fed mice of either sex (data not shown). Thus, hepatic UCP2 does not alter the regulation of glucose homeostasis in lean mice, which express low levels of UCP2.

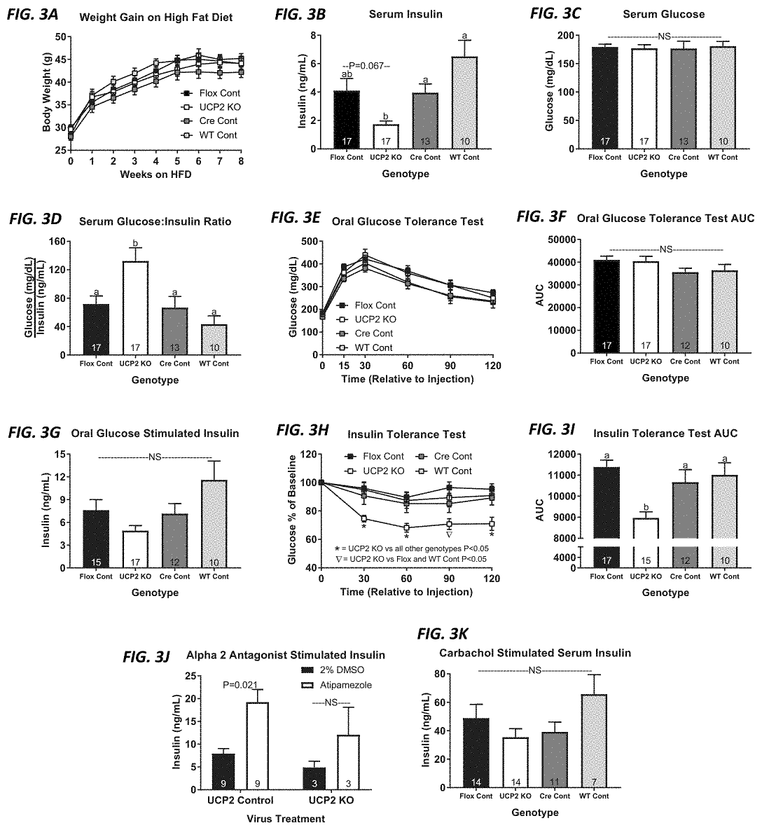

High fat diet (HFD; Teklad, TD 06414) induced similar weight gain across genotypes (see FIG. 3A). Yet, eliminating hepatic UCP2 expression protects against the development of obesity-induced hyperinsulinemia (FIG. 3B). While serum glucose concentrations were comparable among all genotypes, the glucose:insulin ratio was robustly elevated in hepatic UCP2 null mice, indicative of improved insulin sensitivity (FIG. 3C, FIG. 3D). Hepatic UCP2 knockout did not improve glucose tolerance (FIG. 3E, FIG. 3F), perhaps due to an apparent decrease in glucose stimulated serum insulin concentration that did not reach statistical significance (FIG. 3G). All control genotypes were markedly insulin resistant, while hepatic UCP2 knockout mice remained insulin sensitive (FIG. 3H, FIG. 3I). Elimination of hepatic UCP2 did not alter hepatic gluconeogenesis from pyruvate or liver triglyceride accumulation (data not shown).

The change in serum insulin in response to pharmacologically muting the inhibitory signals from the sympathetic nervous system was assessed. The response to carbachol stimulation was also tested to assess sensitivity to the excitatory signals of the parasympathetic nervous system. Both genotypes responded with a rise in serum insulin in response to the alpha 2 adrenergic antagonist, atipamezole. The rise in insulin in response to the muscarinic agonist, carbachol, was similar in control and hepatic UCP2 knockout mice (FIG. 3J, FIG. 3K). Thus, the lack of hyperinsulinemia in the hepatic UCP2 knockout mouse is not mediated by increased activity of the sympathetic nervous system or decreased sensitivity to parasympathetic stimulation.

### Hepatic Hyperpolarization Protects Against Diet-Induced Metabolic Dysfunction

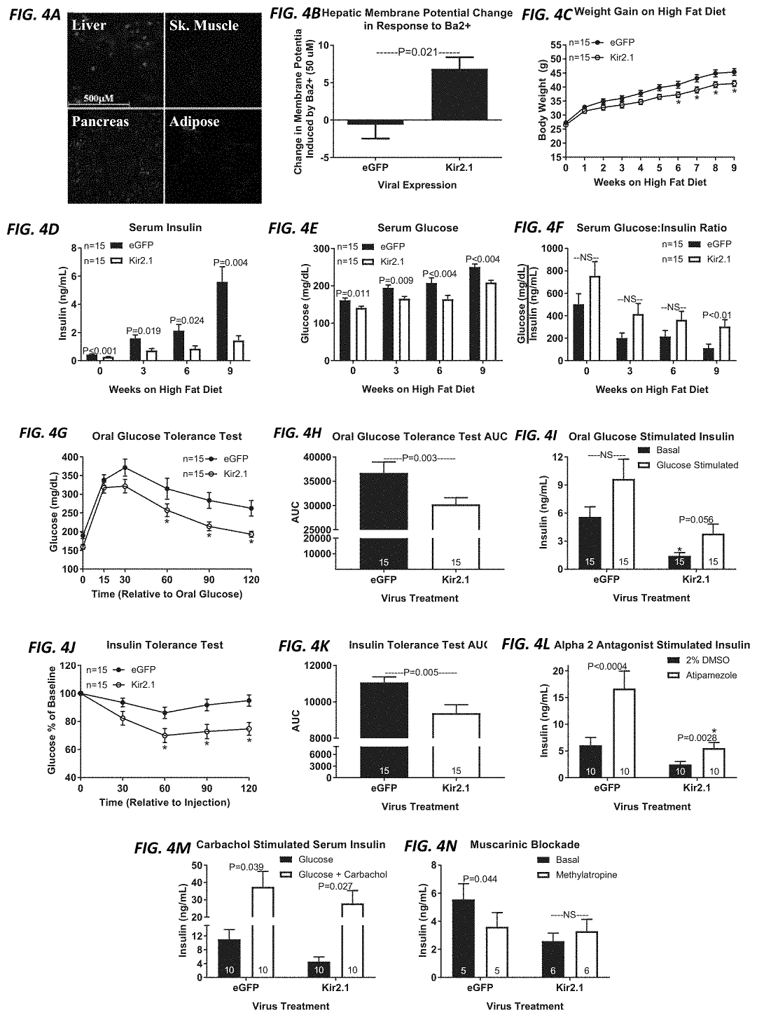

To induce a chronic hyperpolarized state, an AAV8 viral vector encoding TBG promoter driven expression of eGFP and the inward rectifying K⁺ channel, Kir2.1 was used (FIG. 4A). Although this channel is inwardly rectifying in neurons, in hepatocytes with a resting membrane potential that ranges from −15 to −35 mV, Kir2.1 channel expression supports K⁺ efflux and hyperpolarization. The hyperpolarizing effect of Kir2.1 was confirmed by in vivo intracellular measurement of the membrane potential of a hepatocyte before and after bath application of the Kir2.1 antagonist, Barium (Ba²⁺). Ba²⁺ induced a 6.86±1.54 mV depolarization of hepatocytes in Kir2.1 expressing mice, but had no effect (−0.62±1.86 mV) in control eGFP expressing mice (FIG. 4B).

In lean mice that are not hyperinsulinemic, hyperglycemic, glucose intolerant, or insulin resistant, hepatocyte hyperpolarization decreased basal serum insulin and glucose concentrations, improved glucose clearance, and insulin sensitivity (data not shown). This establishes that hepatocyte membrane potential regulates systemic glucose homeostasis in non-disease conditions, and that hepatocyte membrane potential acts as a rheostat that can increase and decrease serum insulin concentrations.

Kir2.1 and eGFP control mice were then placed on a 60% HFD for 9 weeks. Kir2.1 expression depressed weight gain on a HFD, reaching significance from weeks 6-9 on HFD (FIG. 4C). Kir2.1 expression limited the rise in serum insulin and glucose in response to 3, 6, or 9 weeks of HFD feeding (FIG. 4D, FIG. 4E, FIG. 4F). Thus, hepatocyte hyperpolarization protects against the development of hyperinsulinemia and hyperglycemia in diet induced obesity. After 3 weeks on a HFD, Kir2.1 expression continued to improve glucose clearance without altering glucose stimulated serum insulin (data not shown). Insulin tolerance tests reveal comparable insulin sensitivity between Kir2.1 and eGFP expressing mice at 3 weeks of HFD feeding, although Kir2.1 mice show a trend for improved insulin sensitivity (data not shown). After 9 weeks on the HFD, Kir2.1 expression improved glucose tolerance and insulin sensitivity (FIG. 4G, FIG. 4H, FIG. 4I, FIG. 4J, FIG. 4K). Kir2.1 expression also appears to have limited obesity induced hepatic gluconeogenesis, assessed by a pyruvate tolerance test, but did not affect hepatic lipid accumulation on a HFD (data not shown).

The β-cell response to pharmacological manipulation of sympathetic and parasympathetic signaling was tested again. Antagonism of alpha 2 adrenergic receptors and activation of muscarinic receptors (coincident with stimulatory glucose) increased serum insulin independent of Kir2.1 expression (FIG. 4L, FIG. 4M). Thus, the limited hyperinsulinemia in Kir2.1 mice is not a result of increased noradrenergic tone on 1-cells or decreased muscarinic sensitivity.

Cholinergic blockade more profoundly decreases serum insulin concentrations in obese than in lean mice, suggesting that muscarinic signaling at the β-cell is chronically elevated in obesity. Sub-diaphragmatic vagotomy normalizes insulinemia in obese rats by reducing cholinergic action on β-cells. FIG. 4N shows that intraperitoneal methylatropine bromide, a muscarinic receptor antagonist, decreased serum insulin in obese control (eGFP), but not Kir2.1 expressing mice. This indicates that hepatic Kir2.1 expression limits hyperinsulinemia by decreasing parasympathetic acetylcholine signaling onto β-cells.

Hepatocyte Communication with the Hepatic Afferent Vagal Nerve

To investigate potential neurotransmitters that are released by the liver and could affect HVAN firing activity, liver slices were incubated ex vivo and a panel of neurotransmitters released into the media was measured (see Table 1; *Indicates significant difference between obese and lean mice (P<0.05). Data are presented as mean±SEM.).

TABLE 1
Initial neuromodulators panel analysis on media
collected form the liver explant studies.

| Neurotransmitter | Lean | Obese | % Change in |
|---|---|---|---|
| (μmol/μg DNA) | (N = 5) | (N = 3) | Obesity |
| Adenosine | 0.22 ± 0.04 | 0.10 ± 0.01 | −55%* |
| Histidine | 17.74 ± 0.92 | 12.90 ± 0.72 | −27%* |
| Serine | 22.32 ± 3.33 | 13.02 ± 0.53 | −42% |
| Taurine | 238.40 ± 18.41 | 305.18 ± 38.04 | 28% |
| Glutamine | 49.06 ± 5.19 | 40.39 ± 3.98 | −17% |
| Glycine | 130.74 ± 5.16 | 81.31 ± 4.93 | −37%* |
| Aspartic Acid | 6.92 ± 0.55 | 3.47 ± 0.32 | −50%* |
| Glutamic Acid | 30.32 ± 2.12 | 28.74 ± 3.48 | −5.2% |
| GABA | 5.43 ± 0.64 | 8.77 ± 0.53 | 61%* |

Since hepatic lipid accumulation depolarizes hepatocytes, and hepatocyte depolarization decreases HVAN firing activity (FIG. 1A, FIG. 1B), obese livers were expected to display either an increase in the release of inhibitory or a decrease in the release of excitatory neurotransmitters, effectively decreasing the likelihood of triggering an action potential in the HVAN. Hepatocytes from obese mice released more GABA than hepatocytes from lean mice. In turn, Kir2.1 expression decreased obesity induced hepatic slice GABA release. Thus, hepatic lipid accumulation increases release of the inhibitory neurotransmitter GABA, while hyperpolarization reverses this pattern and shifts the release profile back towards that of a lean liver.

Hepatocytes synthesize GABA via the mitochondrial enzyme GABA-Transaminase (GABAT), and obesity increases hepatic GABAT mRNA expression. The reduced state of the mitochondria that results from high β-oxidative activity along with the enhanced gluconeogenic flux in obesity drives the production of GABA. This increase in GABA can act at GABA_{A} receptors on vagal afferents to induce chloride influx and decrease firing rate.

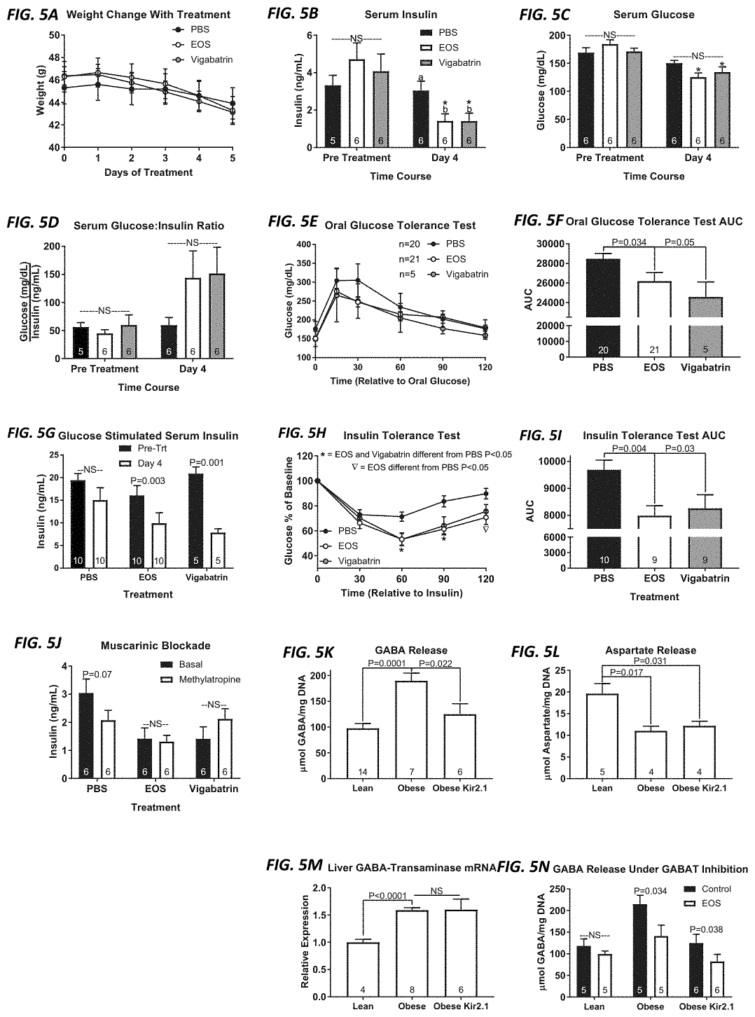

To directly assess the effect of GABAT in obesity induced insulin resistance, hyperinsulinemia, and hyperglycemia, two unique, irreversible GABAT inhibitors, ethanolamino-O-sulphate (EOS) and vigabatrin that reduce hepatic GABAT activity by over 90% within two days were used. EOS does not readily cross the blood brain barrier or decrease central nervous system GABAT activity. Accordingly, the responses to EOS are interpreted to result from peripheral GABAT inhibition. Body weight remained similar among EOS, vigabatrin, and saline injected mice (FIG. 5A). 4 days of EOS or vigabatrin treatment decreased serum insulin and glucose concentrations relative to pre-treatment (FIG. 5B, FIG. 5C). GABAT inhibition (EOS and vigabatrin combined) elevated the glucose:insulin ratio compared to saline mice (P=0.042), although this did not reach significance for the individual inhibitors (FIG. 5D). Glucose clearance and glucose stimulated insulin concentrations were not affected by GABAT inhibition (FIG. 5E, FIG. F, FIG. 5G). However, EOS and vigabatrin improved insulin sensitivity (FIG. 5H). EOS decreased the insulin tolerance test area under the curve as did GABAT inhibition (EOS and vigabatrin; P=0.015; FIG. 5I). Muscarinic blockade tended to decrease serum insulin in saline mice (P=0.07; 32%), while having no effect in EOS or vigabatrin treated mice. The response to methylatropine did differ between control and GABAT inhibitor treated mice, suggesting that these inhibitors limit acetylcholine stimulated hyperinsulinemia (EOS and vigabatrin combined; P=0.024; FIG. 5J).

Referring to FIG. 5K, FIG. 5L, FIG. 5M, and FIG. 5N, hepatic slices were collected from lean, obese, and obese Kir2.1 expressing mice. GABA release (μmol/mg DNA) from hepatic slices is increased by obesity and inhibited by Kir2.1 expression (FIG. 5K). Aspartate release (μmol/mg DNA) is decreased in obesity and not affected by Kir2.1 expression (FIG. 5L). Obesity increases GABA-Transaminase mRNA expression, which is not affected by Kir2.1 expression (FIG. 5M). Bath application of the GABA-T inhibitor, ethanolamine-O-sulfate (EOS), decreased GABA release from slices from obese mice (FIG. 5N).

To determine the duration of GABAT inhibition effects, obese mice were provided with EOS in the drinking water (3 g/L) for 4 days and then monitored during a washout period of 15 weeks. As observed previously, acute EOS treatment decreased serum insulin and glucose concentrations and increased the glucose:insulin ratio relative to pre-treatment values (data not shown). Serum insulin concentrations remained low through 6 weeks washout, but rebounded above pre-treatment concentrations at 15 weeks. EOS improved insulin sensitivity acutely, as mice were insulin resistant again at 2 weeks washout and remained so throughout the 15-week washout period (data not shown). Serum insulin and insulin sensitivity were not affected by EOS or vigabatrin in lean mice (data not shown).

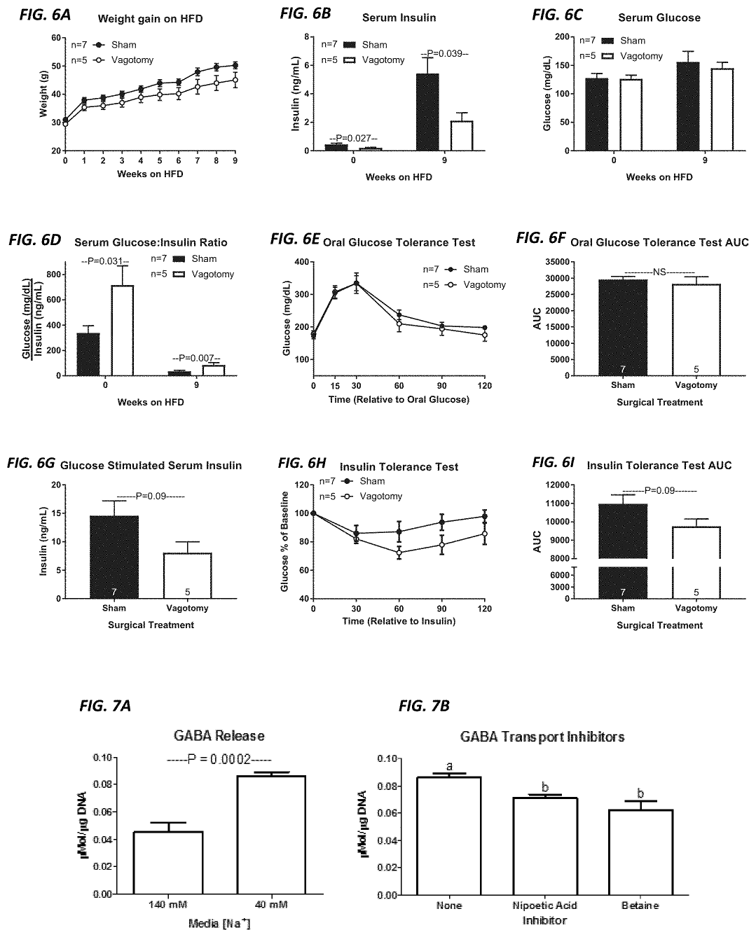

FIG. 6A, FIG. 6B, FIG. 6C, FIG. 6D, FIG. 6E, FIG. 6F, FIG. 6G, FIG. 6H and FIG. 6I show the hepatic vagotomy effects on glucose homeostasis. Hepatic vagotomy limits high fat diet-induced weight gain (FIG. 6A), limits hyperinsulinemia at 9 weeks (FIG. 6B), mutes hyperglycemia in obesity (FIG. 6C), limits oral glucose stimulated insulin release (FIG. 6G), and improves insulin sensitivity (FIG. 6H, FIG. 6H).

FIG. 7A and FIG. 7B show that GABA release from hepatocytes is Na+ dependent and can be inhibited by the GAT2 inhibitor Nipoetic acid and the BGT1 inhibitor Betaine.

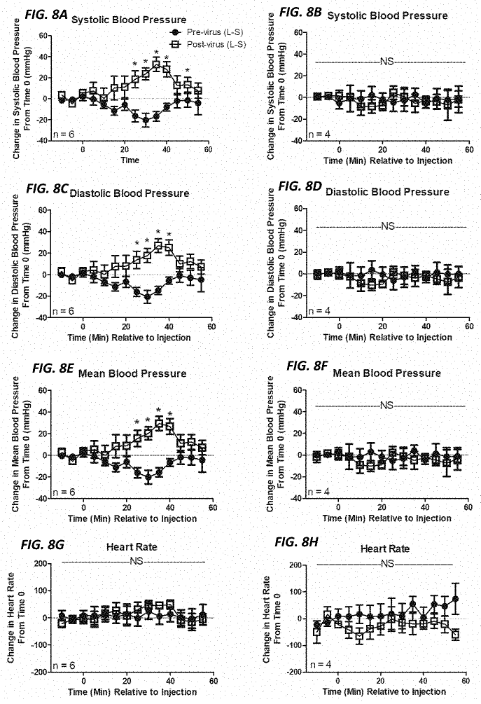

FIG. 8A, FIG. 8B, FIG. 8C, FIG. 8D, FIG. 8E, and FIG. 8F show blood pressure and heart rate data that shows that hepatocyte depolarization increases blood pressure only in mice with an intact hepatic vagal nerve.

### A Hepato-Centric Etiology of Hyperinsulinemia and Insulin Resistance

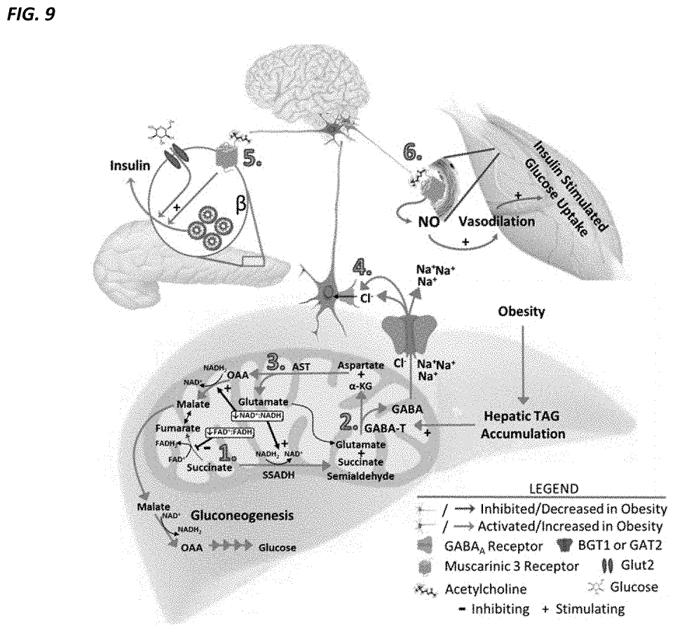

The present invention provides a mechanism by which hepatic lipid accumulation drives the development of hyperinsulinemia and insulin resistance (see FIG. 9). Hepatic lipid accumulation activates PPARα, increasing flux through gluconeogenesis and ketogenesis. Gluconeogenic flux drives hepatic GABA production (FIG. 9; steps 1-3). The ion dependence of GABA transport makes hepatocyte GABA export sensitive to changes in membrane potential. Since GABA transporters are sodium co-transporters, an inability to maintain membrane potential and subsequent intracellular sodium accumulation would be expected to increase GABA export while hepatocyte hyperpolarization would oppose this. Increased hepatic GABA export decreases the firing frequency of the HVAN (FIG. 9; step 4). This decrease in HVAN activity increases pancreatic vagal efferent firing and acetylcholine induced M3R signaling at β-cells (FIG. 9; step 5). When the β-cell is depolarized, including hyperglycemia and obesity, M3R signaling stimulates insulin secretion. Sustained β-cell depolarization in obesity means that elevated acetylcholine signaling persistently encourages insulin release, driving hyperinsulinemia. Acetylcholine signaling at endothelial cells within arterioles stimulates endothelial cell nitric oxide synthase (eNOS) phosphorylation and increases nitric oxide induced vasodilation to enhance insulin sensitivity at skeletal muscle. Insulin normally stimulates skeletal muscle glucose uptake by increasing cell surface Glut4 expression and by stimulating arteriole vasodilation and increasing perfusion. Without wishing to limit the present invention to any theory or mechanism, it is believed that decreased HVAN activity limits parasympathetic efferent outflow to skeletal muscle, promoting insulin resistance (FIG. 9; step 6). Thus, the hepatocyte and vagal nerve independently regulate both insulin release and insulin sensitivity.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although the preferred embodiment of the present invention has been shown and described, it will be readily apparent to those skilled in the art, that modifications may be made thereto which do not exceed the scope of the appended claims. Therefore, the scope of the invention is only to be limited by the following claims. Reference numbers recited in the claims are exemplary and for ease of review by the patent office only, and are not limiting in any way. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

1. A method of treating diabetes, hyperglycemia, insulin resistance, glucose intolerance, or hypertension in a subject in need thereof, said method comprising: administering to the subject a therapeutic amount of a composition for decreasing hepatic GABA synthesis or hepatic GABA release, wherein decreasing hepatic GABA synthesis or hepatic GABA release decreases blood glucose and improves insulin sensitivity.

2. The method of claim 1, wherein the composition prevents obesity-induced depolarization of hepatocytes.

3. The method of claim 1, wherein the composition normalizes blood pressure.

4. The method of claim 1, wherein the composition reduces hepatic mitochondrial uncoupling.

5. The method of claim 1, wherein the composition inhibits activity or expression of GABA-T, BGT1, GAT2, M3R, hepatic succinate semialdehyde dehydrogenase, or UCP2.

6-10. (canceled)

11. The method of claim 1, wherein the composition is a drug, a compound, or a molecule.

12-14. (canceled)

15. The method of claim 1, wherein the composition causes a decrease in blood glucose and a decrease in blood insulin.

16-19. (canceled)

20. The method of claim 1, wherein the composition causes a fasting blood glucose of 120 mg/dL or less and a fasting insulin level of 10 mmol/mL or less.

21. (canceled)

22. The method of claim 1, wherein the composition comprises ethanolamine-O-sulfate (EOS), vigabatrin, or betaine.

23-25. (canceled)

26. A method for improving insulin sensitivity in a subject in need thereof, said method comprising: administering to the subject a therapeutic amount of a composition for decreasing hepatic GABA synthesis or hepatic GABA release, wherein decreasing hepatic GABA synthesis or release improves insulin sensitivity.

27. The method of claim 26, wherein the composition restores insulin sensitivity to that of a non-diabetic individual.

28. The method of claim 26, wherein the composition inhibits activity or expression of hepatic GABA-T, BGT1, GAT2, succinate semialdehyde dehydrogenase, or UCP2.

29-35. (canceled)

36. The method of claim 26, wherein the composition causes a decrease in blood glucose and a decrease in blood insulin.

37. The method of claim 26, wherein the composition causes a fasting blood glucose of 120 mg/dL or less and a fasting insulin level of 10 mmol/mL or less.

38. The method of claim 26, wherein the composition comprises ethanolamine-O-sulfate (EOS), betaine, or vigabatrin.

39-43. (canceled)

44. A method of causing a subject in need thereof to lose weight, said method comprising: administering to the patient a therapeutic amount of a composition for decreasing hepatic GABA synthesis or hepatic GABA release, wherein decreasing hepatic GABA synthesis or hepatic GABA release causes a decrease in food intake so that the subject loses weight.

45. The method of claim 44, wherein the composition prevents obesity-induced depolarization of hepatocytes.

46. The method of claim 44, wherein the composition normalizes blood pressure.

47. (canceled)

48. The method of claim 44, wherein the composition inhibits activity or expression of GABA-T, BGT1, GAT2, succinate semialdehyde dehydrogenase, or UCP2.

49-54. (canceled)

55. The method of claim 44, wherein the composition comprises ethanolamine-O-sulfate (EOS) or vigabatrin.

56-58. (canceled)
